(19) United States
(12) Patent Application Publication
(10) Pub. No.: US 2022/0238204 A1
(43) Pub. Date: Jul. 28, 2022

(54) SYSTEMS AND METHODS TO LINK PSYCHOLOGICAL PARAMETERS ACROSS VARIOUS PLATFORMS

(71) Applicant: Solsten, Inc., St. Paul, MN (US)

(72) Inventors: Joseph Jack Schaeppi, Maple Grove, MN (US); Lynn Danielle Francoise Bergmann, Berlin (DE); Lloyd William West, Berlin (DE)

(21) Appl. No.: 17/157,661

(22) Filed: Jan. 25, 2021

(51) Int. Cl. G16H 20/70; G16H 10/60; G16H 80/00

(52) U.S. Cl. CPC G16H 20/70; G16H 80/00; G16H 10/60

(57) ABSTRACT

Systems and methods to link psychological parameters across various platforms are disclosed. Exemplary implementations may: store, in electronic storage, interaction correlations and psychological profiles; obtain psychological profiles corresponding to users of online platforms; determine similarities between the psychological profiles of the different online platforms; determine, based on the similarities, new interaction correlations between the psychological profiles of the online platforms and content of different ones of the online platforms; store the new interaction correlations to the electronic storage; identify, based on the interaction correlations and/or the new interaction correlations, content for presentation to the users; and effectuate presentation of the content to the users.

### FIELD OF THE DISCLOSURE

The present disclosure relates to systems and methods to link psychological parameters across various platforms.

### BACKGROUND

Users frequently use multiple online platforms. While some of the online platforms and content thereof may be tailored to the users, the customization may be specific to individual users and may be specific to individual online platforms.

### SUMMARY

One aspect of the present disclosure relates to a system configured to link content across a plurality of online platforms based on psychological parameter values of users. Psychological profiles may be obtained from the online platforms of which characterize a user type and/or user. Subsequently, similarities may be determined between the different psychological profiles. Based on the similarities, interaction correlations may be determined or updated. The interaction correlations may indicate content that may suit users that have the different psychological profiles. As such, content from the different platforms and the psychological profiles may be correlated and stored to electronic storage. Based on the interaction correlations, content may be identified for presentation to the users and effectuated. Thus, content on the different platforms may be linked so that users are presented content that suits them based on their psychological profiles and content of other ones of the online platforms.

One aspect of the present disclosure relates to a system configured to link psychological parameters across various platforms. The system may include one or more hardware processors configured by machine-readable instructions, electronic storage, and/or other components. Instruction components may include computer program components. The instruction components may include one or more of profile obtaining component, interaction correlation determination component, content presentation component, user response monitoring component, and/or other instruction components.

The electronic storage may be configured to store interaction correlations, psychological profiles, and/or other information. The individual interaction correlations may indicate content that suits users having the different psychological profiles.

The profile obtaining component may be configured to obtain psychological profiles corresponding to users of online platforms and/or other information. By way of non-limiting illustration, a first set of psychological profiles may be obtained that correspond to users of a first online platform and a second set of psychological profiles may be obtained that correspond to users of a second online platform. The interaction correlations may indicate content that suits users having the first set of psychological profiles on the first online platform. The interaction correlations may indicate content that suits users having the second set of psychological profiles on the second online platform.

The interaction correlation determination component may be configured to determine similarities between the psychological profiles of the different online platforms. By way of non-limiting illustration, similarities may be determined between individual ones of the psychological profiles in the first set of psychological profiles and individual ones of the psychological profiles in the second set of psychological profiles.

The interaction correlation determination component may be configured to determine new interaction correlations between the psychological profiles of the online platforms and content of different ones of the online platforms. Determination of the new interaction correlations may be based on the similarities. By way of non-limiting illustration, a first new interaction correlation may be determined between the psychological profiles in the first set of psychological profiles and content of the second platform. The interaction correlation determination component may be configured to store the new interaction correlations to the electronic storage.

The content presentation component may be configured to identify content for presentation to the users. The content may be identified based on the interaction correlations and/or the new interaction correlations. By way of non-limiting illustration, the first set of psychological profiles may include a first psychological profile that corresponds to a first user of the first online platform. Content for presentation to the first user may be identified based on the first new interaction correlation. The content presentation component may be configured to effectuate presentation of the content to the users and/or other information. By way of non-limiting illustration, presentation of the content to the first user may be effectuated.

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates a system configured to link psychological parameters across various platforms, in accordance with one or more implementations.

FIG. 2 illustrates a method to link psychological parameters across various platforms, in accordance with one or more implementations.

FIG. 3A-C illustrates an example implementation of the system configured to link psychological parameters across various platforms, in accordance with one or more implementations.

### DETAILED DESCRIPTION

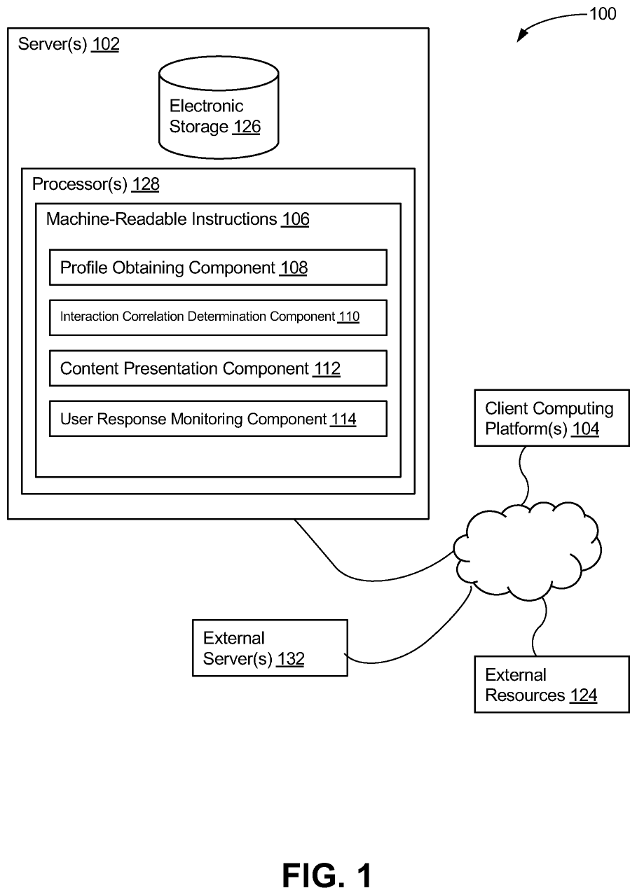

FIG. 1 illustrates a system 100 configured to link psychological parameters across various platforms, in accordance with one or more implementations. In some implementations, system 100 may include one or more servers 102. Server(s) 102 may include an electronic storage 126 and/or other components. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Users may access system 100 via client computing platform(s) 104.

In some implementations, external server(s) 132 may be configured to provide and/or host one or more online platforms. External server(s) 132 may refer to an individual (video) game provider, a gaming console, game companies, website developers, user experience companies, and/or a combination thereof. As used herein, “online platform” or variations thereof may include a digital experience. A digital experience may include one or more of a game (e.g., online via web browser, via desktop application, via mobile application, via game console, etc.), a website (e.g., retailers), a mobile application, a desktop application, a social network platform, a service provider (e.g., E-mail provider), and/or other digital experiences. Such digital experiences may collect similar information related to its users and provide some of the same interactions or similar interactions between individual users, between the individual users and virtual content, and/or other interactions. Virtual content (or pieces thereof) may include one or more of game content, social media content, publicity content, and/or other virtual content. The game content may include one or more of a player-controlled character, a non-player-controlled character, a task, a quest, an assignment, a mission, a level, a chapter, a mini-game, a virtual item, a virtual resource (e.g., weapon, tool), an in-game power, an in-game skill, an in-game technology, and/or other pieces of game content. By way of non-limiting example, virtual items may include one or more of clothing, pets, transportation units (e.g., aircrafts, motor vehicles, watercrafts, etc.), units, buildings, and/or other virtual items. The social media content and the publicity content may include one or more of an image, a video, a description, a financial component (e.g., for purchasing, selling, trading, donating, etc.), and/or other content. The online platforms may be accessed by the users via client computing platform(s) 104.

Server(s) 102 may be configured by machine-readable instructions 106. Machine-readable instructions 106 may include one or more instruction components. The instruction components may include computer program components. The instruction components may include one or more of profile obtaining component 108, interaction correlation determination component 110, content presentation component 112, user response monitoring component 114, and/or other instruction components.

Electronic storage 126 may be configured to store interaction correlations, psychological profiles, and/or other information. The psychological profiles may characterize a user type and/or a particular user. The psychological profile may define measured psychological parameter values to psychological parameters that characterize the user type and/or the particular user. A user type may be defined by multiple psychological parameter values that characterize how a user interacts with the online platforms, why the user interacts with particular online platforms, when the user interacts with the particular online platforms, and/or other user type information. The psychological profiles may characterize particular individual users such as a first user, a second user, and so on. The particular user may have a user type characterized by one of the psychological profiles.

The psychological parameter values may define, by way of non-limiting example, achievement motivation, motivation, personality inventory, cultural values, competitiveness, positive and negative affect before, during, and/or after engagement with the online game (i.e., emotions), communication style, personal values, daily routines/activities, life/gaming pain points, life/gaming hopes and aspirations, wellbeing, user experience, gaming/experience using time, subscription behavior, affinity information, personality, emotional style, goal orientation, goal commitment, ego and task orientation, relatedness, sense of community, social influence, social identity, group identification, we-identity, quality of life, satisfaction with life, work-related quality of life, mindfulness, happiness, emotional intelligence, self-awareness/internal awareness, external awareness, connectedness to nature, social connectedness, social bonding, perceived stress, depression, anxiety, decision-making style, thinking style, critical thinking, cognitive approach to learning, learning style, attributional style, internality-externality, stability-instability, global-specific, creativity, curiosity, playfulness, exploration, mental strength, grit, flourishing, gratitude, inspiration, spirituality, hedonism, materialism/material values, perceptions, sentiments, and/or other psychological parameters.

Achievement motivation may include compensatory effort, competitiveness, confidence in success, dominance, eagerness to learn, engagement, fearlessness, flexibility, flow, goal setting, independence, internality, persistence, preference in difficult tasks, pride in productivity, self-control, status orientation, ambition, self-assurance, and/or other psychological parameters. Motivation may include mastery, purpose, autonomy, and/or other psychological parameters.

Personality inventory may include neuroticism, openness, conscientiousness, extraversion, and agreeableness and/or other psychological parameters. Neuroticism may include anxiety, impulsiveness, vulnerability, and/or other psychological parameters. Openness may include fantasy, feelings/empathy, action, and/or other psychological parameters. Conscientiousness may include achievement striving, competence, self-discipline, and/or other psychological parameters. Extraversion may include warmth assertiveness, activity, and/or other psychological parameters. Agreeableness may include trust, altruism, modesty, and/or other psychological parameters.

Cultural values may include individualism, indulgence, long term orientation, masculinity, power distance, uncertainty avoidance, and/or other psychological parameters. Competitiveness may include avoidant, collaborative, competitive affectivity, dependent, dominant, general competitiveness, independent, personal enhancement, and/or other psychological parameters.

Positive and negative affect before, during, and/or after engaging in the online game may include hostility, joviality, negative emotions, positive emotions, sadness, self-assurance, and/or other psychological parameters. Communication style may include feeler, intuitor, sensor, thinker, and/or other psychological parameters.

Wellbeing may include social wellbeing, psychological wellbeing, physical wellbeing, physical activity, sleep, bounded reciprocity, resilience grit, and/or other psychological parameters.

Personality may include anger, hostility, depression, self-conscientiousness, excitement-seeking, positive emotions, gregariousness, ideas, values, aesthetics, tender-mindedness, straightforwardness, compliance, deliberation, order, dutifulness, and/or other psychological parameters.

Emotional style may include resilience, outlook, social intuition, self-awareness, sensitivity to context, attention, and/or other psychological parameters.

Goal orientation may include mastery approach/learning goal orientation, performance approach/performance goal orientation, performance avoid/avoidance goal orientation, and/or other psychological parameters.

Work-related quality of life may include structure, boundaries, focus, efficiency, information provision, communication, psychological support, stress at/from work, psychological safety, connectedness with team, motivation to work, adaptability, job/career satisfaction, control at work, home-work interface, general wellbeing, working conditions, and/or other psychological parameters.

Mindfulness may include observing, describing, acting with awareness, non-judgment, non-reactivity, and/or other psychological parameters.

Emotional intelligence may include emotion perception, emotion expression, emotion management, emotion regulation, impulse control, relationships, stress management, and/or other psychological parameters.

Social connectedness may include social connectedness, loneliness, membership self-esteem, private self-esteem, public self-esteem identity self-esteem, interdependent self, independent self, social avoidance, social distress, and/or other psychological parameters.

Decision-making style may include respected, confident, spontaneous, dependent, vigilant, avoidant, brooding, intuitive, anxious, and/or other psychological parameters.

Thinking style may include intuitive, experiential, analytical, rational, and/or other psychological parameters.

Cognitive approaches to learning may include avoidant, participative, competitive, collaborative, dependent, independent, and/or other psychological parameters.

Learning style may include visual (spatial), aural (auditory-musical), verbal (linguistic), physical (kinesthetic), logical (mathematical), social (interpersonal), solitary (intrapersonal), and/or other psychological parameters.

Mental strength may include tenacity, confidence, optimism, adaptability, self-awareness, reliability, responsibility, well-being, and/or other psychological parameters.

Flourishing may include positive emotion, engagement, relationships, meaning, accomplishment, health, loneliness, and/or other psychological parameters.

The psychological parameter values of the psychological parameters may be a number score on a predetermined range unique to each psychological parameter, a letter score, and/or other type of value than may characterize intensity of the psychological parameter that the psychological parameter value defines and thus a particular user as whole. The intensity may represent significance of what a given psychological parameter of the particular user is.

In some implementations, the psychological profiles may define user behavior patterns of the users. User behavior patterns may include actions performed by the users within a virtual space. The actions may include one or more of a purchase, a sale, a trade, a selection of a user interface element, time spent on a particular user interface, time of a session of the online game, engagement with virtual content, communication of the users with particular users, completion of tasks by the users or lack thereof, failure of tasks by the users, frequent interaction with content of the users, formation of alliances by the users, and/or other actions performed by the users. The user behavior patterns may include individual ones of the actions, sets of actions, ordered sets of actions, and/or multiple of the individual actions, the sets of actions, and the ordered set of actions.

The psychological profiles may define performance information that includes counts of the user behavior patterns, time information of the user behavior patterns, occurrence of the user behavior patterns, duration of the user behavior patterns, a time period over which the user behavior patterns occurred (e.g., over one week, over one month, over 100 hours, etc.), and/or other performance information. The time information may include, by way of non-limiting example, time of day of the user behavior patterns, day of the week of the user behavior patterns, date of the user behavior patterns, successiveness of the user behavior patterns, whether it the user behavior patterns are a reaction, and/or other time information.

The virtual space may provide an online game and/or other digital experiences and may comprise a simulated space that is accessible by users via clients (e.g., client computing platform(s) 104) that present the views of the virtual space to a user. The simulated space may have a topography, express ongoing real-time interaction by one or more users, and/or include one or more objects positioned within the topography that are capable of locomotion within the topography. In some instances, the topography may be a 2-dimensional topography. In other instances, the topography may be a 3-dimensional topography. The topography may include dimensions of the space, and/or surface features of a surface or objects that are “native” to the space. In some instances, the topography may describe a surface (e.g., a ground surface) that runs through at least a substantial section of the space. In some instances, the topography may describe a volume with one or more bodies positioned therein (e.g., a simulation of gravity-deprived space with one or more celestial bodies positioned therein). The instance executed by the computer components may be synchronous, asynchronous, and/or semi-synchronous.

The above description of the manner in which views of the virtual space are provided is not intended to be limiting. The virtual space may be expressed in a more limited, or richer, manner. For example, views determined for the virtual space may be selected from a limited set of graphics depicting an event in a given place within the virtual space. The views may include additional content (e.g., text, audio, pre-stored video content, and/or other content) that describes particulars of the current state of the place, beyond the relatively generic graphics. For example, a view may include a generic battle graphic with a textual description of the opponents to be confronted. Other expressions of individual places within the virtual space are contemplated.

The individual interaction correlations may indicate content that suits users having the different psychological profiles. The content that suits the users have the different psychological profiles may entertain, interest, inspire, encourage, and/or otherwise may be appropriate for the user type and/or the particular user. It will be appreciated that the description herein of “correlations” between psychological parameters and content which are positively correlated is not intended to be limiting, and that negative correlations between psychological parameters and content are also contemplated, and may be included in the generic “correlations”. The determination of negative correlations may be made in cases where users characterized by a psychological profile strongly presenting a psychological parameter avoid specific content, and/or where users characterized by a psychological profile that do not present the psychological parameter respond to the specific content relatively more (e.g., in frequency, total performances, etc.) than users characterized by the psychological profile that strongly present the psychological parameter.

By way of non-limiting example, the content may include one or more of a character, a game, a game asset, a recommendation, a suggestion, a promotion, and/or other content. The character may refer to an object (or group of objects) present in a virtual space that corresponds to an individual user (e.g., an avatar) and/or are controlled by the user. In some implementations, the character may not correspond to an individual user but rather provide information (e.g., the recommendation, the suggestion) to the user. The game asset may include a virtual item, a virtual resource (e.g., weapon, tool), of in-game powers, in-game skills, in-game technologies, and/or other game assets. The recommendation may include a particular selection and/or action that the user is advised to select and/or do. The suggestion may include particular ideas, plans, and/or strategies for the user to consider executing, following, and/or is determined they will enjoy. The promotion may include discount codes, coupons, bonuses, and/or other promotions of the virtual items, products, and/or services that the user may utilize. By way of non-limiting example, products and/or services may relate to beauty (e.g., skincare, makeup), home improvement, decoration, clothing, accessories, technology, kitchen, and/or other categories. In some implementations, the content may be particularly related to the online platform that presents it or may not be related to the online platform that presents it. In some implementations, the content of the online platforms may already be correlated to the psychological profiles corresponding to the users of the respective online platforms.

Profile obtaining component 108 may be configured to obtain psychological profiles corresponding to users of online platforms and/or other information. In some implementations, the psychological profiles may be obtained from the online platforms. In some implementations, the online platforms may transmit the psychological profiles that correspond to its users to profile obtaining component 108. In some implementations, the profile obtaining component 108 may be configured to obtain the psychological profiles at particular times. The particular times may include at conclusion of a session by individual users with the online platforms, periodically (e.g., every minute, every 5 minutes, every 30 minutes, etc.) throughout the session, at the end of a day, and/or other time. A session may refer to a period of time spent on the online platforms by the individual users.

By way of non-limiting illustration, a first set of psychological profiles may be obtained that correspond to users of a first online platform and a second set of psychological profiles may be obtained that correspond to users of a second online platform. The interaction correlations may indicate content that suits users having the first set of psychological profiles on the first online platform. Furthermore, the interaction correlations may indicate content that suits users having the second set of psychological profiles on the second online platform. The first platform and the second platform may be separate and distinct online platforms.

Interaction correlation determination component 110 may be configured to determine similarities between the psychological profiles of the different online platforms. By way of non-limiting illustration, similarities may be determined between individual ones of the psychological profiles in the first set of psychological profiles and individual ones of the psychological profiles in the second set of psychological profiles. In some implementations, determining similarities may include determining similarities between psychological parameter values of the psychological profiles of the different online platforms. In some implementations, determining the similarities may include determining psychological parameter values that are the same and/or psychological parameter values that are within a particular range. Ranges may be predefined, modified, and/or may vary per psychological parameter. For example, a range of psychological parameter values for the collaborative psychological parameter may be 1-10 where values 1-2 are considered similar, values 3-4 are considered similar, values 5-7 are considered similar, and values 8-10 are considered similar. A first psychological profile included in the first set of psychological profiles may define a 5 for collaborative (i.e., the psychological parameter). A second psychological profile included in the second set of psychological profiles may define a 7 for collaborative. Thus, the collaborative psychological parameter for the first psychological profile and the second psychological profile are determined to be similar. In some implementations, determining similarities may include comparing the psychological parameter values and/or other comparison techniques.

In some implementations, determining similarities between the psychological profiles of the different online platforms may include determining similarities between the user behavior patterns of the psychological profiles. Such determination may include comparing the user behavior patterns defined by the psychological profiles of the different platforms and/or other comparison techniques. For example, comparing the user behavior patterns may include determining which actions, sets of actions, or ordered actions are common and/or the same between the psychological profiles and the performance information thereof, and/or other determinations of similarities.

Interaction correlation determination component 110 may be configured to determine new interaction correlations between the psychological profiles of the online platforms and content of different ones of the online platforms. Determination of the new interaction correlations may be based on the similarities. New interaction correlations may refer to interaction correlations that have not been determined or identified by interaction correlation determination component 110 and are thus not stored in electronic storage 126. By way of non-limiting illustration, a first new interaction correlation may be determined between the psychological profiles in the first set of psychological profiles and content of the second platform. In some implementations, interaction correlation determination component 110 may be configured to improve/update the interaction correlations stored in electronic storage 126. Updating the interaction correlations may include revising the content suits the users having the different psychological profiles, adding content, removing content, revising the psychological parameter values, revising the user behavior patterns, and/or other updates. In some implementations, the interaction correlations may be updated subsequent to the determinations of similarities and/or at particular times (e.g., periodically, after individual sessions, etc.).

Interaction correlation determination component 110 may be configured to store the new interaction correlations, the updates to the interaction correlations, and/or other information to electronic storage 126. The interaction correlations and updates thereof may be store indefinitely, for a predefined period of time, a period of time specified by an administrator of system 100, and/or other amount of time.

Content presentation component 112 may be configured to identify content for presentation to the users. The content may be identified based on the interaction correlations and/or the new interaction correlations. That is, the content indicated by the interaction correlations may be identified for presentation. In some implementations, content presentation component 112 may be configured to obtain the interaction correlations from the online platforms prior to identifying the content for presentation to the users. The content may be stored by the different online platforms (i.e., external server(s) 132 thereof), by electronic storage 126, both, and/or by external resources 124. In some implementations, content presentation component 112 may have access to the external server(s) 132 of the online platforms to identify the content on behalf of the online platforms. The access may be set as limited, all-inclusive, or modifiable from the external server(s) 132. The content for presentation to the users may be identified for the online platform the users are interacting with.

By way of non-limiting illustration, the first set of psychological profiles may include the first psychological profile that corresponds to the first user of the first online platform. The first user may be interacting with the first online platform. Content for presentation to the first user may be identified based on the first new interaction correlation. The content for presentation to the first user is at least some of the content of the second online platform. Thus, upon the first user visiting the second online platform, the identified content of the second online platform may be presented via the second online platform.

In some implementations, user response monitoring component 114 may be configured to monitor the user responses of the users to the content on the different online platforms. By way of non-limiting illustration, user responses of the users having the first set of psychological profiles (e.g., a first user of the first online platform) content presented on the second online platform may be monitored.

In some implementations, the user response may be reactions of the users to the content presented on the second online platform. By way of non-limiting example, the user response may include selection of the presented content by the users, purchases by the users, communication between the users, continued engagement with the second online platform, the individual actions, the sets of actions, the ordered set of actions and/or multiples thereof, and/or other user responses. As such, identifying the content for presentation to the user and/or determination of additional ones of the interaction correlations may be based on the user response.

Content presentation component 112 may be configured to effectuate presentation of the content to the users and/or other information. The content and/or the other information may be presented via the online platforms and via user interfaces of client computing platform(s) 104 associated with the users. By way of non-limiting illustration, presentation of the content to the first user may be effectuated. In some implementations, the content identified may be communicated via the network with the external server(s) 132 of the online platforms the external server(s) 132 to present.

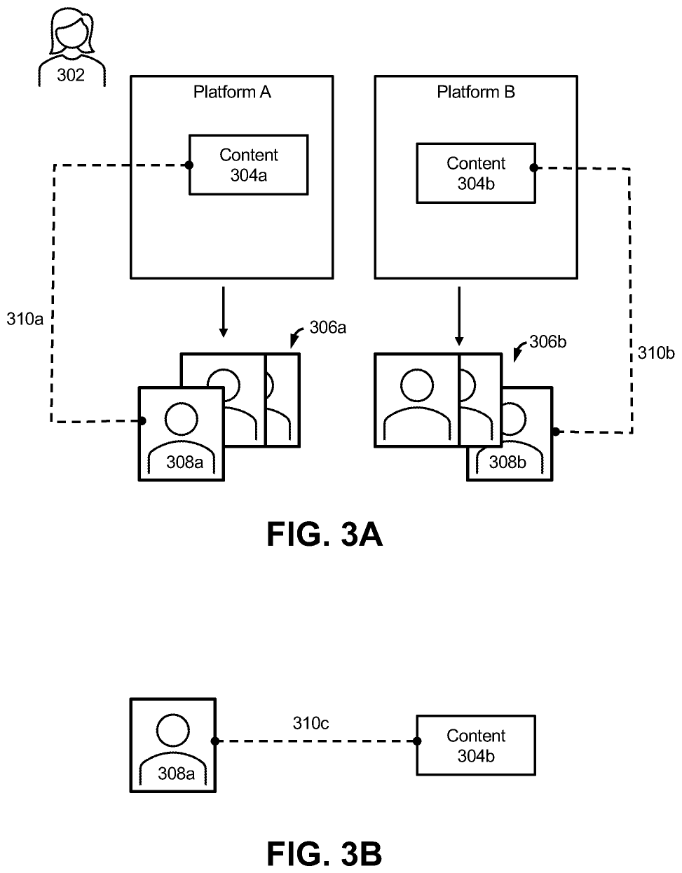

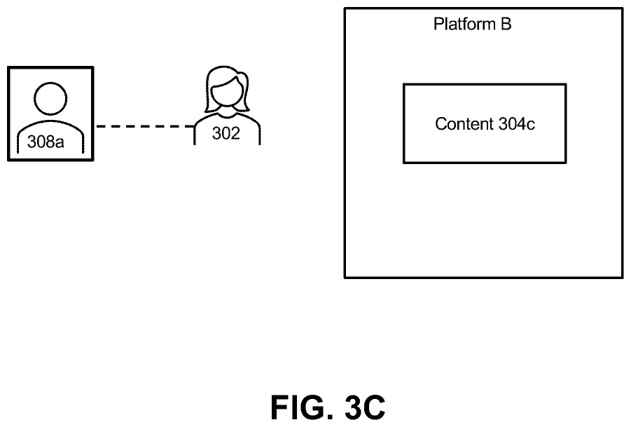

FIG. 3A-C illustrates an example implementation of the system configured to link psychological parameters across various platforms, in accordance with one or more implementations. FIG. 3A may illustrate an (online) Platform A and an (online) Platform B that a user 302 may interact with. Platform A may present a content 304a and Platform B may present a content 304b. A set 306a of psychological profiles may be obtained that corresponds to user of Platform A by system 100 (of FIG. 1). A set 306b of psychological profiles that correspond to users of Platform B may be obtained by system 100. Set 306a may include a psychological profile 308a and set 306b may include a psychological profile 308b. An interaction correlation 310a between psychological profile 308a and content 304a may be stored where content 304a is indicated to suit users having psychological profile 308a. Likewise, an interaction correlation 310b between psychological profile 308b and content 304b may be stored where content 304b is indicated to suit users having psychological profile 308b. Psychological profiles 308a and 308b may have similarities determined between them. Referring to FIG. 3B, based on the similarities, interaction correlation 310c may be determined between psychological profile 308a and content 304b. Meaning, content 304b may further suit users having psychological profile 308a.

Referring to FIG. 3C, Platform B (the same as FIG. 3A) may present a content 304c for user 302 or other users having psychological profile 308a. Content 304c may be identified based on interaction correlation 310c of FIG. 3B and may be related to Platform B.

In some implementations, server(s) 102, client computing platform(s) 104, and/or external resources 124 may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which server(s) 102, client computing platform(s) 104, and/or external resources 124 may be operatively linked via some other communication media.

A given client computing platform 104 may include one or more processors configured to execute computer program components. The computer program components may be configured to enable an expert or user associated with the given client computing platform 104 to interface with system 100 and/or external resources 124, and/or provide other functionality attributed herein to client computing platform(s) 104. By way of non-limiting example, the given client computing platform 104 may include one or more of a desktop computer, a laptop computer, a handheld computer, a tablet computing platform, a NetBook, a Smartphone, a gaming console, and/or other computing platforms.

External resources 124 may include sources of information outside of system 100, external entities participating with system 100, and/or other resources. In some implementations, some or all of the functionality attributed herein to external resources 124 may be provided by resources included in system 100.

Server(s) 102 may include electronic storage 126, one or more processors 128, and/or other components. Server(s) 102 may include communication lines, or ports to enable the exchange of information with a network and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 126 may comprise non-transitory storage media that electronically stores information. The electronic storage media of electronic storage 126 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 126 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 126 may include one or more virtual storage resources (e.g., cloud storage, a virtual private network, and/or other virtual storage resources). Electronic storage 126 may store software algorithms, information determined by processor(s) 128, information received from server(s) 102, information received from client computing platform(s) 104, and/or other information that enables server(s) 102 to function as described herein.

Processor(s) 128 may be configured to provide information processing capabilities in server(s) 102. As such, processor(s) 128 may include one or more of a digital processor, an analog processor, a digital circuit designed to process information, an analog circuit designed to process information, a state machine, and/or other mechanisms for electronically processing information. Although processor(s) 128 is shown in FIG. 1 as a single entity, this is for illustrative purposes only. In some implementations, processor(s) 128 may include a plurality of processing units. These processing units may be physically located within the same device, or processor(s) 128 may represent processing functionality of a plurality of devices operating in coordination. Processor(s) 128 may be configured to execute components 108, 110, 112, and/or 114, and/or other components. Processor(s) 128 may be configured to execute components 108, 110, 112, and/or 114, and/or other components by software; hardware; firmware; some combination of software, hardware, and/or firmware; and/or other mechanisms for configuring processing capabilities on processor(s) 128. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 108, 110, 112, and/or 114 are illustrated in FIG. 1 as being implemented within a single processing unit, in implementations in which processor(s) 128 includes multiple processing units, one or more of components 108, 110, 112, and/or 114 may be implemented remotely from the other components. The description of the functionality provided by the different components 108, 110, 112, and/or 114 described below is for illustrative purposes, and is not intended to be limiting, as any of components 108, 110, 112, and/or 114 may provide more or less functionality than is described. For example, one or more of components 108, 110, 112, and/or 114 may be eliminated, and some or all of its functionality may be provided by other ones of components 108, 110, 112, and/or 114. As another example, processor(s) 128 may be configured to execute one or more additional components that may perform some or all of the functionality attributed below to one of components 108, 110, 112, and/or 114.

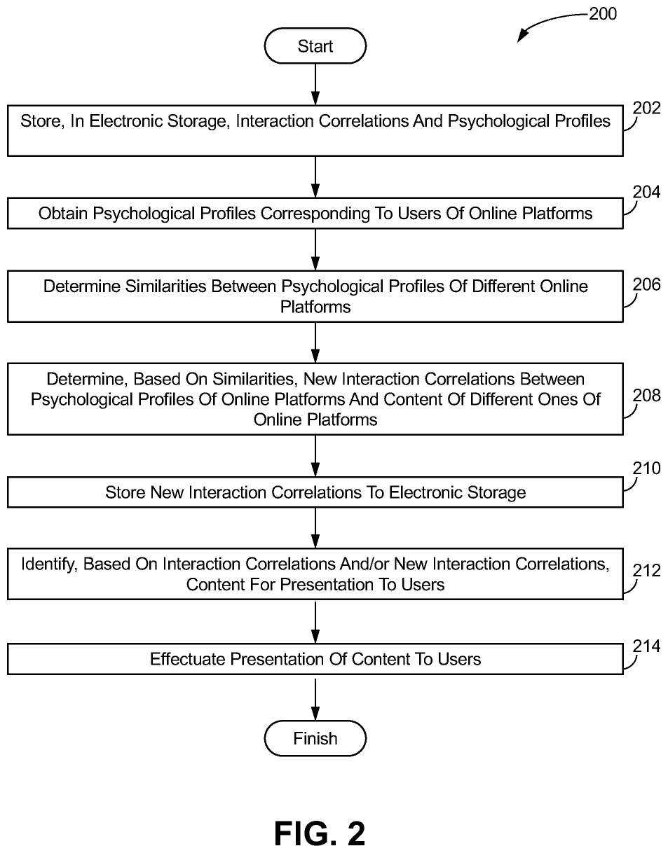

FIG. 2 illustrates a method 200 to link psychological parameters across various platforms, in accordance with one or more implementations. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

An operation 202 may include storing, in electronic storage, interaction correlations and psychological profiles. The individual interaction correlations may indicate content that suits users having the different psychological profiles. Operation 202 may be performed by electronic storage 122, in accordance with one or more implementations.

An operation 204 may include obtaining psychological profiles corresponding to users of online platforms. Operation 204 may be performed by one or more hardware processors configured by machine-readable instructions including a component that is the same as or similar to profile obtaining component 108, in accordance with one or more implementations.

An operation 206 may include determining similarities between the psychological profiles of the different online platforms. Operation 206 may be performed by one or more hardware processors configured by machine-readable instructions including a component that is the same as or similar to interaction correlation determination component 110, in accordance with one or more implementations.

An operation 208 may include determining, based on the similarities, new interaction correlations between the psychological profiles of the online platforms and content of different ones of the online platforms. Operation 208 may be performed by one or more hardware processors configured by machine-readable instructions including a component that is the same as or similar to interaction correlation determination component 110, in accordance with one or more implementations.

An operation 210 may include storing the new interaction correlations to the electronic storage. Operation 210 may be performed by one or more hardware processors configured by machine-readable instructions including a component that is the same as or similar to interaction correlation determination component 110, in accordance with one or more implementations.

An operation 212 may include identifying, based on the interaction correlations and/or the new interaction correlations, content for presentation to the users. Operation 212 may be performed by one or more hardware processors configured by machine-readable instructions including a component that is the same as or similar to content presentation component 112, in accordance with one or more implementations.

An operation 214 may include effectuating presentation of the content to the users. Operation 214 may be performed by one or more hardware processors configured by machine-readable instructions including a component that is the same as or similar to content presentation component 112, in accordance with one or more implementations.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

1. A system configured to link psychological parameters across various platforms, the system comprising:
electronic storage configured to store interaction correlations and psychological profiles, wherein the individual interaction correlations indicate content that suits users having the different psychological profiles, wherein individual ones of the psychological profiles define measured psychological parameter values to psychological parameters that characterize a user type and/or a particular user, wherein individual ones of the psychological parameters are associated with value ranges that define similar psychological parameter values, wherein the psychological parameters include openness, conscientiousness, extraversion, agreeableness, neuroticism, communication style, learning style, thinking style, satisfaction with life, connectedness to nature, quality of life, mindfulness, curiosity, exploration, gratitude, spirituality, materialism, and/or cultural values; and
one or more processors configured by machine-readable instructions to:
obtain, from online platforms, psychological profiles corresponding to users of digital experiences provided by the online platforms, wherein the online platforms are individually provided by distinct game providers and/or web developers to provide different ones of the digital experiences, wherein the digital experiences include at least a game, an e-commerce website, and/or a social media digital space, such that a first set of psychological profiles are obtained that correspond to users of a first online platform and a second set of psychological profiles are obtained that correspond to users of a second online platform, wherein the first set of psychological profiles includes a first psychological profile that corresponds to a first user of the first online platform and the second set of psychological profiles includes a second psychological profile that corresponds to the first user of the second online platform, wherein the first psychological profile includes at least a first psychological parameter value to a first psychological parameter and the second psychological profile includes at least a second psychological parameter value to the first psychological parameter, wherein the interaction correlations indicate content that suits users having the first set of psychological profiles on the first online platform, and wherein the interaction correlations indicate content that suits users having the second set of psychological profiles on the second online platform;
determine whether the psychological parameter values to the psychological parameters defined by the psychological profiles of the different online platforms are within the same value range to determine similarities between the psychological profiles, such that at least a determination of whether the first psychological parameter value and the second psychological parameter value are within the same value range associated with the first parameter is made;
determine, based on the similarities, new interaction correlations between the psychological profiles of the online platforms and content of different ones of the online platforms such that a first new interaction correlation is determined between the first psychological profile and content of the second platform correlated with the second psychological profile;
store the new interaction correlations to the electronic storage;
identify, based on the interaction correlations and/or the new interaction correlations, content for presentation to the users such that content for presentation to the first user is identified based on the first new interaction correlation; and
effectuate presentation of the content to the users via the online platforms such that presentation of the content to the first user is effectuated.

2. The system of claim 1, wherein the one or more processors are further configured by the machine-readable instructions to:
monitor user response of the users having the first set of psychological profiles to content presented on the second online platform,
wherein identifying the content for presentation to the users is based on the user response.

3. (canceled)

4. (canceled)

5. (canceled)

6. The system of claim 1, wherein the content for presentation to the users is identified for the online platform the users are interacting with such that the content for presentation to the first user is content of the second online platform and presented via the second online platform.

7. The system of claim 2, wherein the user response is a reaction of the users to the presented content on the second online platform, wherein the user response includes selection of the presented content by the users, purchases by the users, communication between the users, and/or continued engagement with the second online platform.

8. The system of claim 1, wherein the one or more processors are further configured by the machine-readable instructions to obtain the interaction correlations from the online platforms.

9. A method configured to link psychological parameters across various platforms, the method comprising:
storing, in electronic storage, interaction correlations and psychological profiles, wherein the individual interaction correlations indicate content that suits users having the different psychological profiles, wherein individual ones of the psychological profiles define measured psychological parameter values to psychological parameters that characterize a user type and/or a particular user, wherein individual ones of the psychological parameters are associated with value ranges that define similar psychological parameter values, wherein the psychological parameters include openness, conscientiousness, extraversion, agreeableness, neuroticism,
communication style, learning style, thinking style, satisfaction with life, connectedness to nature, quality of life, mindfulness, curiosity, exploration, gratitude, spirituality,
obtaining, from online platforms, psychological profiles corresponding to users of digital experiences provided by the online platforms, wherein the online platforms are individually provided by distinct game providers and/or web developers to provide different ones of the digital experiences, wherein the digital experiences include at least a game, an e-commerce website, and/or a social media digital space, such that a first set of psychological profiles are obtained that correspond to users of a first online platform and a second set of psychological profiles are obtained that correspond to users of a second online platform, wherein the first set of psychological profiles includes a first psychological profile that corresponds to a first user of the first online platform and the second set of psychological profiles includes a second psychological profile that corresponds to the first user of the second online platform, wherein the first psychological profile includes at least a first psychological parameter value to a first psychological parameter and the second psychological profile includes at least a second psychological parameter value to the first psychological parameter, wherein the interaction correlations indicate content that suits users having the first set of psychological profiles on the first online platform, and wherein the interaction correlations indicate content that suits users having the second set of psychological profiles on the second online platform;
determining whether the psychological parameter values to the psychological parameters defined by the psychological profiles of the different online platforms are within the same value range to determine similarities between the psychological profiles, such that at least a determination of whether the first psychological parameter value and the second psychological parameter value are within the same value range associated with the first parameter is made;
determining, based on the similarities, new interaction correlations between the psychological profiles of the online platforms and content of different ones of the online platforms such that a first new interaction correlation is determined between the first psychological profile and content of the second platform correlated with the second psychological profile;
storing the new interaction correlations to the electronic storage;
identifying, based on the interaction correlations and/or the new interaction correlations, content for presentation to the users such that content for presentation to the first user is identified based on the first new interaction correlation; and
effectuating presentation of the content to the users via the online platform such that presentation of the content to the first user is effectuated.

10. The method of claim 9, further comprising:
monitor user response of the users having the first set of psychological profiles to content presented on the second online platform,
wherein identifying the content for presentation to the users is based on the user response.

11. (canceled)

12. (canceled)

13. (canceled)

14. The method of claim 9, wherein the content for presentation to the users is identified for the online platform the users are interacting with such that the content for presentation to the first user is content of the second online platform and presented via the second online platform.

15. The method of claim 10, wherein the user response is a reaction of the users to the presented content on the second online platform, wherein the user response includes selection of the presented content by the users, purchases by the users, communication between the users, and/or continued engagement with the second online platform.

16. The method of claim 9, further comprising obtaining the interaction correlations from the online platforms.
